(19) United States
(12) Patent Application Publication
(10) Pub. No.: US 2026/0240440 A1
(43) Pub. Date: Aug. 20, 2026

(54) WEARABLE FITNESS DEVICE WITH DYNAMIC VISUAL FEEDBACK

(71) Applicant: EPOC Life, LLC, Pacific Palisades, CA (US)

(72) Inventor: David Dodd O'Neal, JR., Pacific Palisades, CA (US)

(21) Appl. No.: 19/540,504

(22) Filed: Feb. 13, 2026

Related U.S. Application Data
(60) Provisional application No. 63/758,953, filed on Feb. 14, 2025.

(51) Int. Cl. A61B 5/024; A61B 5/00

(52) U.S. Cl. CPC A61B 5/024; A61B 5/4866; A63B 2230/06

(57) ABSTRACT

Apparatuses, systems, and techniques to display exercise information on a wearable fitness device. In at least one embodiment, a wearable fitness device comprises one or more display surfaces to display one or more visual indicators of exercise performance, where the one or more display surfaces are selected and/or configured to maximize non-foveal perception of the one or more visual indicators.

### CLAIM OF PRIORITY

This application claims the benefit of U.S. Provisional Application No. 63/758,953, entitled “WEARABLE FITNESS DEVICE WITH DYNAMIC VISUAL FEEDBACK,” filed Feb. 14, 2025, the entire contents of which is incorporated herein by reference.

### BACKGROUND

Devices that provide fitness information include smartwatches or smartphones with visual displays and interfaces that provide a wide variety of information to the user. However, techniques for providing this information to a user during and after fitness activities can be improved.

### BRIEF DESCRIPTION OF THE DRAWINGS

Various techniques will be described with reference to the drawings, in which:

FIG. 1 illustrates aspects of a wearable fitness device to display exercise information, in accordance with an embodiment;

FIG. 2 illustrates segments of a segmented flexible band to display exercise information, in accordance with an embodiment;

FIG. 3 illustrates an example process of displaying EPOC information on a wearable fitness device, in accordance with an embodiment.

FIG. 4 illustrates an example process of displaying exercise information on a flexible band, in accordance with an embodiment;

FIG. 5 illustrates an example embodiment of a wearable fitness device with a flexible band to display exercise information, in accordance with at least one embodiment;

FIG. 6 illustrates an example embodiment of a wearable fitness device with a flexible band to display exercise information via an ambient or off-axis visualization, in accordance with at least one embodiment;

FIG. 7 illustrates an example of a wearable fitness device to display exercise information as a secondary display, in accordance with an embodiment;

FIG. 8 illustrates an example process of generating visual indications to reduce the cognitive burden of perceiving an exercise performance metric, in accordance with an embodiment;

FIG. 9 illustrates an example process of generating a visual indication of an exercise performance metric, in accordance with an embodiment;

FIG. 10 illustrates aspects of a wearable fitness device to display exercise information using side-oriented display surfaces, in accordance with an embodiment; and

FIG. 11 illustrates a software application to generate visual signals for non-foveal perception on a smartwatch.

### DETAILED DESCRIPTION

In the preceding and following description, various techniques are described. For purposes of explanation, specific configurations and details are set forth in order to provide a thorough understanding of possible ways of implementing the techniques. However, it will also be apparent that the techniques described below may be practiced in different configurations without the specific details. Furthermore, well-known features may be omitted or simplified to avoid obscuring the techniques being described.

In an example embodiment, a wearable fitness device comprises one or more display surfaces that are designed to maximize peripheral or off-axis viewing of exercise performance indicators. While traditional wearable devices typically have high-resolution screens that enable the detailed display of information, they also typically require the user of the device to interact closely with the device, and correspondingly require a high degree of cognition on the user's part in order to perceive the indicator. Examples of this involve requiring the user to raise and position the device so that the screen can be looked at directly, using foveal focus. In contrast, embodiments of the disclosed wearable fitness device utilize display surfaces that are arranged and controlled in a manner that avoids the need to rely on foveal vision, and instead are optimized for peripheral, off-axis, and/or non-foveal vision. A benefit of this approach is that the user is enabled to perceive visual indicators on the wearable fitness device without altering the performance of their exercise. For example, the wearable fitness device's display surfaces may be arranged and/or controlled in a manner that allows a runner to perceive the indicator during a normal stride or gait cycle.

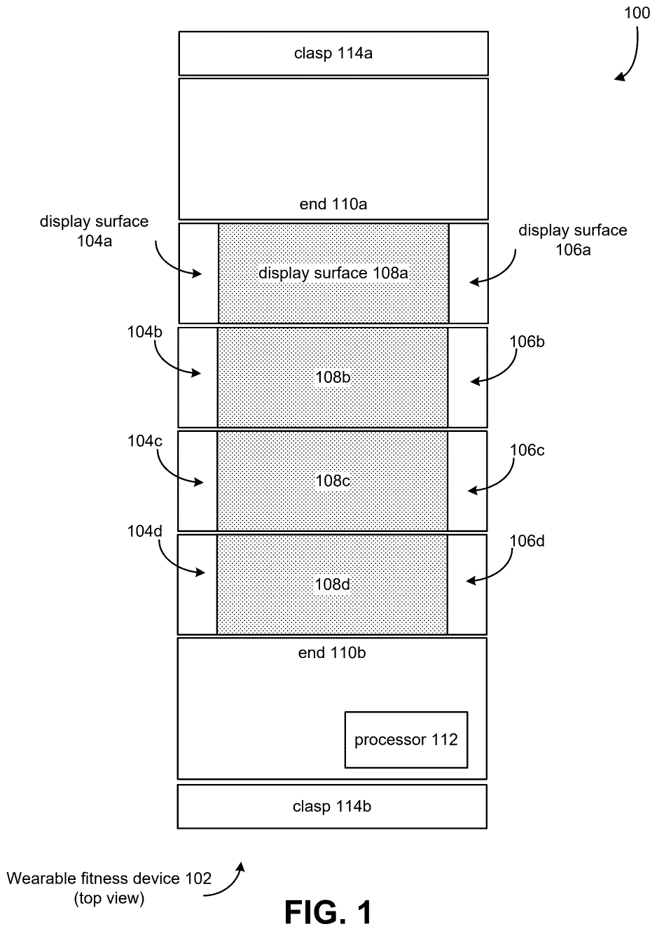

FIG. 1 illustrates aspects of a wearable fitness device 102 to display exercise information, in accordance with an embodiment. The example 100 of FIG. 1 depicts a wearable fitness device 102 comprising multiple display surfaces. In this example, the display surfaces are a plurality of display surfaces 104a-d on a first edge of the wearable fitness device 102, a plurality of display surfaces 106a-d on a second edge of the wearable fitness device, and a plurality of display surfaces 108a-d on a middle portion of the wearable fitness device 102. The flexible band may also comprise portions, such as ends 110a, b of the wearable fitness device 102, that do not comprise display surfaces. Some embodiments may include fewer display surfaces, such as embodiments that only include the middle portion of display surfaces, or only that only include the display surfaces on one or two edges of the wearable fitness device. Embodiments may include a plurality of display surfaces over at least a majority of the flexible display band, e.g. by including one or more display surfaces that span at least 50% of the length of the display band.

A display surface, such as any of the depicted display surfaces 104a-d, 106a-d, 108a-d, can include displays incorporating LED, MicroLED, LCD, OLED, AMOLED, QLED, E-Ink, or other similar display types, including high or low resolution displays capable of displaying complex graphics. In some embodiments, each display surface is a simple LED or other lighting system, such as a color and/or brightness-configurable LED light.

In an embodiment, the display surfaces function as a peripheral optical band or ambient optical interface, meaning that they are configured to diffuse light into a continuous visualization that is easily visible via peripheral, off-axis, or non-foveal vision. The diffusion may be accomplished by using one or more techniques such as embedded scattering particles, volume diffusion, surface texturing, surface-relief, and transmission through translucent materials. For example, in an embodiment, the display surfaces 1004, 1006 comprise a light source, such as an LED, that is transmitted through a material to generate a diffuse lighting effect that is prominently visible to the user. In an embodiment, the material is a transparent, semi-transparent, or translucent silicone band. In other embodiments, a glass or acrylic material is used. In other embodiments, surface texturing on the band 1016 causes light diffusion to emphasize a visual indication on a portion of the band that is perceptible via peripheral, off-axis, or non-foveal vision.

A flexible band 102 can comprise a continuous or segmented material or device that can be worn on the wrist or arm. This material or device may described herein as a flexible band. The display surfaces 104a-d, 106a-d, 108a-d may be mounted on or embedded in the flexible band. For example, in some embodiments the display surfaces 104a-d, 106a-d, 108a-d are LED lights embedded within a translucent, transparent, or semi-transparent material. In other embodiments, the display surfaces 104a-d, 106a-d, 108a-d are mounted on or underneath the flexible band.

The wearable device 102 may further comprise one or more processors, such as processor 112, as well as memory (not depicted) to store non-transitory processor-executable instructions to perform various functions as described herein. In an embodiment, the processor 112 is embedded in or affixed to an end 110b of the flexible band 102.

The wearable device 102 may further comprises one or more clasps 114a,b to secure the wearable device 102 around the wrist or arm of a user. A clasp 114a,b may include any of a variety of devices to secure the wearable device 102 in this manner. For example, a clasp 114a,b may include a buckle, deployant clasp, butterfly clasp, fold-over clasp, sliding buckle, or any other similar mechanism. In some embodiments, the one or more clasps 114a, b include sensors or other devices to detect that the wearable device 102 has been secured on the wrist or arm of a user. In some embodiments, the one or more clasps 114a,b include sensors that enable detection of the circumference of the user's arm or wrist. In some embodiments, these sensors further enable detection of the orientation of the wearable device and the corresponding display surfaces 104a-d, 106a-d, 108a-d on the user's wrist. Here, orientation can include the relative position of the various display surfaces 104a-d, 106a-d, 108a-d. For example, if the clasps 114a, b are secured on a relatively small wrist, the positions of the respective display surfaces 104a-d, 106a-d, 108a-d may be different than if the clasps 114a,b were secured on a relatively large wrist. The orientation and/or circumference information may then be used, by processor 112, to infer which display surfaces are most easily visible to the user. Some embodiments may also use additional sensor information, such as accelerometers or positioning sensors, to determine circumference and/or orientation information.

Note that although FIG. 1 describes each display surface as being a separate device, such as a separate individual LEDs, other embodiments may use display surfaces that are implemented on a single device, such as a higher-resolution LED screen. In such cases, the LED screen may be divided into zones, where each zone can correspond to one of the display surfaces described above.

In at least one embodiment, a wearable fitness device 102 comprises a band worn around a user's wrist or arm. The band may comprise one or more display surfaces configured to maximize perception by the wearer, particularly for off-axis viewing and scenarios where it is desirable to avoid the need for the user to rely on foveal vision to perceive a visual performance indicator. In at least one embodiment, the band is accompanied by a smartwatch that performs aspects of the processes described herein. In at least one embodiment, the band of the wearable fitness device is joined to or otherwise integrated with the smartwatch.

In at least one embodiment, the wearable fitness device provides maximized off-axis viewing of a runner's pace and heart rate.

In at least one embodiment, the wearable fitness device 102 receives information indicating desired splits, and generates visual indications (for example, by changing color and/or brightness) to indicate to the user whether they are on pace or not, according to the user-provided splits.

In at least one embodiment, the wearable fitness device 102 comprises one or more of a microcontroller including a processor, a Bluetooth low-energy chip or other means of wireless communication with a smartwatch or other computing device, a battery, a charging port or charging interface, and one or more display surfaces. Embodiments of the one or more display surfaces are described herein.

In at least one embodiment, the wearable fitness device 102 is configured to include diffusion material to cause light emitted from the display surfaces to be diffused. The diffusion material helps to diffuse the emitted light, so as to enable the emitted light to be more readily perceived off-axis, e.g. without the user needing to rely on foveal vision to perceive the corresponding visual indication.

In at least one embodiment, the wearable fitness device 102 determines applicable performance metrics and drives a display of the wearable fitness device. This can comprise causing the display surfaces of the wearable fitness device 102 to display green when a metric is within a target range, red when the metric is above the target range, and blue when the metric is below the target range. It will be appreciated that other combinations of ranges and colors may be appropriate, and may be different for different types of exercise metrics.

In at least one embodiment, the wearable fitness device 102 includes an audio mode, that can be used to provide context for visual signals without requiring glancing at the screen. For example, an audio alert may be generated when the colors displayed by the wearable fitness device change. For example, the audio alert might be a message such as “Pace too high. Slow down” to provide advise relevant to a displayed visual indication.

In at least one embodiment, a user can toggle between modes that reflect instant pace (e.g., within an exercise interval) and average pace (e.g., for marathon goal times).

In at least one embodiment, the wearable fitness device 102 provides information related to how well a user adheres to a target goal. This may be referred to, in some embodiments, as a pulse score. For example, in one mode of operation, the wearable fitness device during exercise may indicate a green color when currently matching a target pace, and one or more other colors when outside the range. In another mode of operation, the wearable fitness device can display a simple score or color that corresponds to the percentage of time the user has spent in the “green” zone. This can assist the user in determining whether they conformed to the planned workout schedule.

In at least one embodiment, the wearable fitness device 102 displays a smoothness metric. This can highlight volatility in metrics such as pace. In a smartwatch application, smoothness can be indicated in a visual display that includes a target band and highlights periods in which performance was inside, above, or below that band. On the wearable fitness device, a visual indicator can indicate green (for example) when metrics volatility is acceptably low.

In at least one embodiment, the device generates, or causes to be generated, alerts or messages (e.g., a “milestone card”) when a user achieves a positive exercise trend. The milestone card can further include specific physiological explanations for the improvement, e.g. “Your heart is pumping more blood per beat,” “You're burning fuel more efficiently,” “Your consistent Zone 2 work has increased your Mitochondrial Density,” or “Oxygen is reaching your muscles faster. You have likely increased Capillary Density in your legs.” In at least one embodiment, these milestone cards are automatically sent or posted, e.g. to a social media account. In some embodiments, an interface with as messaging or social media account is accessed on a smartwatch or other computing device.

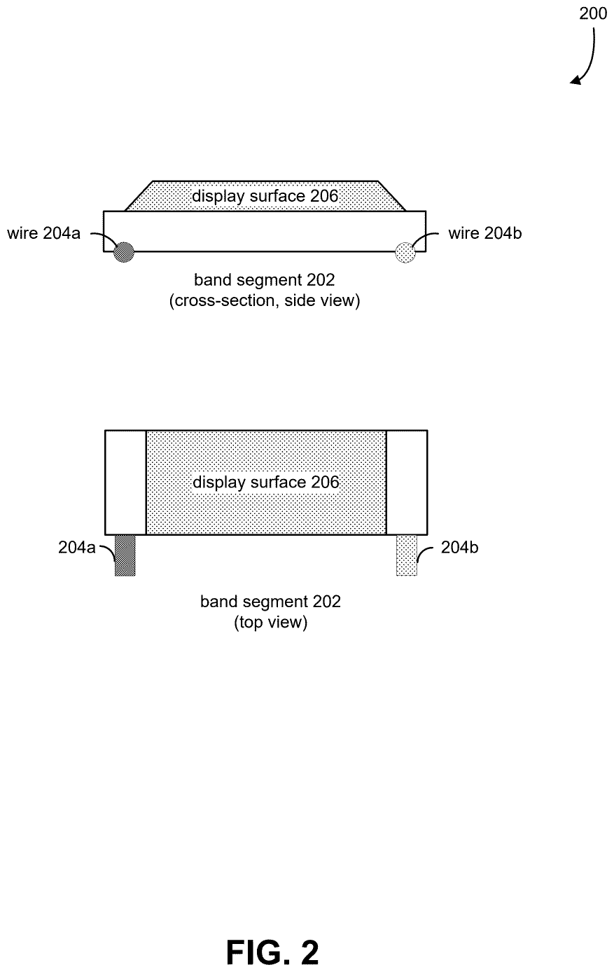

FIG. 2 illustrates segments of a segmented flexible band to display exercise information, in accordance with an embodiment. In this example embodiment 200, a wearable device, such as the wearable device 102 of FIG. 1, includes a segmented flexible band, where each band-segment 202 comprises at least one display surface 206. Each band segment 202 may further comprise one or more wires 204a, b to connect to other band segments and/or to a processor and related components, such as to the processor 112 depicted in FIG. 1 and/or another other component required to control the display surface 206. In embodiments, the display surface 206 may be controlled, by the processor 112, independently of other display surfaces that may be included in the wearable fitness device 102.

In an embodiment, a wearable fitness device provides real-time visual feedback on heart rate zones, post-exercise metabolic activity, training performance, and other physiological or exercise-related data through a flexible, high-visibility display system. The display system may include, for example, a LED, OLED, e-ink, or other current or future flexible display technologies, adapted to be worn on the wrist or arm.

In an embodiment, the wearable fitness device receives heart rate data from a system component, and dynamically translates this data into visual feedback via a display system utilizing color-coded indicators, pulsation effects, and adaptive brightness for real-time performance tracking. This system component, in some embodiments, is external to the wearable fitness device, and may for example be a smart watch (such as an Apple Watch), a pulse monitoring device, a smartphone, or other device capable of capturing physiological information. In an embodiment, the wearable fitness device is a Pulse Afterburn Band.

In an embodiment, a companion application, such as a WatchOS or a companion mobile application, processes heart rate data to calculate Excess Post-Exercise Oxygen Consumption (EPOC) and uses the calculations to compute training insights, post-workout calorie expenditure estimates, and fatigue monitoring. The processed data is then provided (for example by transmission) to the wearable fitness device, which visually represents the insights, such as insights regarding post-exercise afterburn effect, through dynamic, adaptive display feedback, as described in more detail herein. This functionality enables users to operate the wearable fitness device independently of devices comprising complex, expensive, or comparatively heavy displays. For example, the wearable fitness device may, in some embodiments, be used with only a smart watch or other external heart rate monitoring device.

EPOC includes effects related to the increased rate of oxygen intake following strenuous activity. EPOC may be referred to as an afterburn or post-exercise effect that impacts the body's physiological processes after exercise. This may include effects on oxygen debt, metabolic rate, and caloric burn. For example, during intense exercise, the human body may use more oxygen than it can take in, resulting in a deficit of oxygen within the body. EPOC may therefore include processes that repay this deficit. Regarding metabolic rate, the body's metabolic rate may be elevated following exercise while it replenishes energy stores and clears lactate from the bloodstream. Caloric burn rate also tends to be elevated after exercise. The duration and intensity of exercise influence the extent of EPOC. For example, high-intensity workouts tend to result in a more significant and prolonged EPOC effect compared to more moderate or low-intensity exercises. Embodiments include software and displays integrated into a wearable fitness device to maximize user understanding of effects related to EPOC. This can include either relevant effects during exercise, and/or relevant effects after exercise.

In an embodiment, sensors collect biometric or physiological data, which can include, for example, heart rate, body temperature, electrocardiogram data, and other such information. Sensors may also collect other exercise-related information, such as accelerometer or global-positioning system (“GPS”) information.

In an embodiment, these sensors are located in a smartwatch or smartphone, and data collected from the sensors is transmitted to the wearable fitness device. In other embodiments, these sensors are integrated into the wearable fitness device, which directly collects and uses the information. In still other embodiments, the wearable fitness device collects some information uses one or more integrated sensors, and receives other information that is collected from one or more sensors on an external device, such as a smartphone, and transmitted to the wearable fitness device.

In an embodiment, the wearable fitness device features a magnetic clasp mechanism that powers the device on when secured, eliminating the need for a manual power button, and is designed to conform to a variety of wrist and arm wear positions.

In an embodiment, the wearable fitness device includes as part of its display system, a multi-surface display system enables that different data sets (e.g., heart rate on one surface, pace or cadence on another) to be accessed via wrist or arm rotation, allowing hands-free metric tracking.

In an embodiment, an AI-driven coaching system in the companion (e.g., a WatchOS or mobile companion application) delivers real-time audio guidance ensuring users stay within optimal heart rate zones, adjust pacing dynamically, and reach specific training benchmarks.

In an embodiment, a novel metabolic visualization system is implemented in the wearable fitness device, to allow users to see their post-workout calorie burn progression through real-time, color-coded feedback. Using this aspect, the wearable fitness device enhances training efficiency, heart rate zone adherence, and post-workout metabolic awareness, offering an intuitive, hands-free, and behaviorally adaptive fitness solution.

In an embodiment, a user-configurable display setting allows users to customize which metrics are displayed on different locations of the band via a companion application, providing the ability to switch between configurations.

In an embodiment, a portion of a wearable fitness is configured to accept user input. For example, a portion of the wearable fitness device include a pressure sensor or pressure-activated switch. The user input may, in some embodiments, be used to conveniently switch between display modes, to adjust brightness, to indicate exercise-related information, and so forth. In an embodiment, one or more zones of the wearable fitness device comprise these sensors and are configured to accept such user input, and comprise these or other suitable sensors or switches. In some embodiments, these sensors or switches are distributed uniformly across some or all of the surface of the wearable fitness device.

These and other embodiments of the present disclosure may incorporate heart rate zone training, which is an effective method for improving cardiovascular endurance, optimizing fat burn, and enhancing athletic performance. However, existing fitness wearables primarily focus on data collection and post-workout analysis, and thus fail to provide real-time, glanceable heart rate zone feedback that enables users to make immediate adjustments during exercise. Current solutions are reliant on small smartwatch screens that require the user to disrupt the natural flow of their workout to check them. Furthermore, regarding EPOC (Excess Post-Exercise Oxygen Consumption), no wearable device currently visually represents the magnitude or duration of EPOC in real-time. This deficiency leaves users unaware of their post-exercise metabolic impact. Further, wearable devices currently fail to transform complex metabolic data into an intuitive, glanceable visualization. Current devices are limited to small screens that fail to actively influence user behavior through real-time training guidance and post-workout motivation, in many cases due to a failure to adapt to human behavior, anatomy, or preferences during exercise.

Additionally, current devices do not feature adaptive display systems that dynamically adjust to different wrist and arm sizes and positions. Current fitness wearables also do not integrate a seamless, clasp-powered activation mechanism that eliminates the need for manual power controls.

These and other embodiments address these deficiencies by providing hardware, software, and/or other devices to provide a real-time visualization of physiological data, including in some embodiments a user's heart rate and a novel visualization of EPOC (the afterburn effect). By integrating a visual display, AI coaching prompts, and a multi-surface display where heart rate and performance metrics (such as pace or cadence) are positioned on multiple surfaces easily visible during or after exercise, users are able to instantly view different data points by naturally adjusting their wrist orientation, eliminating the need for manual screen navigation.

In an embodiment, the wearable fitness device delivers real-time, 360° visual feedback on heart rate zones, post-exercise metabolic activity, and training performance through a flexible, high-visibility display. This may include, but is not necessarily limited to, display systems that use LED, OLED, e-ink, flexible displays, or other display technologies, integrated into a wearable band designed for wrist or arm placement. Unlike traditional fitness wearables that rely on small, limited screens, embodiments of the wearable fitness device system may feature a continuous, wraparound display system that fully encircles the wrist or arm, creating a highly glanceable, immersive visualization system for real-time performance monitoring.

In an embodiment, the wearable fitness device system acts as a display mechanism, receiving heart rate data from an external source, such as an Apple Watch, and translating it into dynamic, color-coded visual feedback. A companion application enables advanced user customization, real-time coaching, and post-workout metabolic tracking. The companion application calculates Excess Post-Exercise Oxygen Consumption (EPOC) by analyzing workout intensity, time spent in various heart rate zones, and heart rate recovery rates. The wearable fitness device system then visually represents the afterburn effect post-workout by modulating colors and pulsation frequency on the wearable fitness device's display, to reflect estimated caloric afterburn duration and intensity.

In an embodiment, a wearable fitness device computes and displays EPOC information using a dynamic visual display in which color ranges correspond to caloric afterburn ranges. In some embodiments, these colors and/or ranges are customizable. In some embodiments, these colors and/or ranges are dynamically adjusted.

This wearable fitness device features a multi-surface display system on the band of the device, and causes distinct performance metrics to be displayed on various locations of the band, allowing users to instantly access different data points by adjusting their wrist or arm orientation, without requiring manual screen navigation.

For example, the top portion of the band may show heart rate, while the bottom display may show pace, cadence, or speed. Through the companion app, users can manually configure which metrics appear on each area of the band, selecting from preset training modes or assigning custom data layouts. In some embodiments, these positions are dynamically updated to account for a position of the device on the user's wrist or arm, or to account for a position of the wrist or arm.

In an embodiment, a wireless synchronization module performs real-time updates between the companion application and the wearable fitness device. The wearable fitness system uses this information to serve as a 360° post-workout metabolic visualization system, capable of displaying the EPOC afterburn effect across all or almost all of the exterior surface of the band. In an embodiment, the color intensity and pulsation rate displayed on the band gradually decrease post-workout, and thereby visually represent the body's metabolic cooldown process in real time. Unlike traditional wearables that provide only numerical EPOC estimates, this system gamifies post-exercise recovery by offering a highly intuitive, glanceable representation of ongoing calorie burn.

In an embodiment, the wearable fitness device incorporates a magnetic clasp mechanism that powers the device on when secured and off when unclasped, eliminating the need for a manual power button.

In an embodiment, an AI-driven coaching system delivers real-time audio guidance helping users maintain optimal heart rate zones, adjust pacing, and optimize training goals based on historical performance data. In an embodiment, this AI-driven coaching system is a part of the companion application, while in other embodiments, it is integrated into the wearable fitness device itself.

In an embodiment, the wearable fitness device provides a novel metabolic visualization system that allows users to see their post-workout calorie burn progression through a real-time, color-coded, display system. This display enhances training efficiency, heart rate zone adherence, and post-workout metabolic awareness, offering an intuitive, customizable, and behaviorally adaptive fitness solution for endurance athletes, HIIT participants, and fitness enthusiasts.

In an embodiment, a wearable fitness device comprises a 360° high-visibility display for heart rate and performance metrics. This wearable fitness device comprises a flexible display system and a component for driving the flexible display system.

In an aspect of this embodiment, the flexible display system is a flexible, continuous, wraparound display system. This may include, but is not necessarily limited to, displays using LED, OLED, e-ink, and/or flexible display technology. The display system may further be designed to conform to various wrist and arm sizes. The display may further span from 80-100% of the wearable fitness device's circumference. The display may be used to visually represents a wide range of performance metrics, including but not limited to real-time heart rate zones, pace, cadence, speed, exertion levels, recovery status, fatigue indicators, and post-exercise metabolic activity, through dynamic visual patterns and adaptive feedback. The display may further provide uninterrupted, glanceable feedback from multiple angles, allowing users to monitor relevant performance data without looking at a smartwatch or smartphone screen, regardless of placement on the wrist or arm.

In an aspect of this embodiment, the component for driving the flexible display system comprises a multi-surface display, wherein at least two distinct display areas show different real-time metrics, including but not limited to heart rate, speed, cadence, pace, or EPOC-based caloric burn. This component may further allows for customization of displayed metrics across multiple surfaces, including potential additional display areas based on future user configurations. This component may further allow users to instantly access different data points by adjusting wrist and/or arm orientation rather than tapping or swiping on a touchscreen. Users may also choose to display only one metric across the entire band.

In an aspect of this embodiment, the wearable fitness device further comprises a component to allow for a user to configure a display setting that allows users to customize which metrics are displayed on each surface via a companion application, with the option for automatic reconfiguration based on workout mode, real-time performance metrics, or historical user behavior.

In an aspect of this embodiment, the wearable fitness device further comprises a component to provide for adjustments to brightness and color distribution based on the user's wrist size, ensuring consistent readability across different wrist circumferences.

In an aspect of this embodiment, the wearable fitness device further comprises a magnetic clasp mechanism that automatically powers the device on when secured and off when unclasped, eliminating the need for a manual power button.

In an aspect of this embodiment, the wearable fitness device further comprises a wireless communication module enabling the wearable device to receive heart rate and performance data from an external device.

In an embodiment, a wearable fitness device provides adaptive heart rate zone coaching, and comprises a heart rate monitoring component, a display, and a component to generate coaching information. The heart rate monitoring component may comprise sensors or receivers to obtain and process heart rate data to be used to determine the user's current training zone. The display may be a dynamic visual display system that may be caused to display pulses in distinct colors and intensities to indicate whether the user should increase or decrease effort to maintain their target heart rate zone. The component to generate coaching information may comprise a processor and/or software to implement AI-driven coaching. The AI-driven coaching may generate real-time training feedback. The feedback may then be provided to the user via audio, haptic feedback, visual cues, vibrations, or other indications perceptible to the user. These indications may guide users in maintaining or improving training performance.

In an aspect of this embodiment, and in other embodiments, audio prompts may be used. Examples of these include, but are not limited to, messages such as “Increase effort to reach Zone 4,” “You need two more minutes in Zone 5 to optimize EPOC,” or “Your heart rate is dropping too quickly—adjust your pacing.”

In an aspect of this embodiment, the wearable fitness device comprises a smart fatigue management system that monitors heart rate variability (HRV) and recovery indicators to prevent overtraining, providing alerts when rest is recommended.

In an aspect of this embodiment, the wearable fitness device comprises a processor and/or software to implement a progress-tracking algorithm that compares real-time workout performance to historical benchmarks, issuing motivational cues to help users surpass prior achievements.

In an embodiment, a wearable fitness device comprises components to visualize post-workout metabolic activity. This embodiment may comprise a software application that calculates Excess Post-Exercise Oxygen Consumption (EPOC) by analyzing workout intensity, time spent in each heart rate zone, and heart rate decay rate post-exercise.

This embodiment may further comprise a display system that uses color-coding to visually represents post-exercise metabolic activity by mapping estimated caloric afterburn ranges onto the device's display, wherein blue represents an estimated 0 -50 calories burned post-workout, green represents 51-100 additional calories, purple represents 101-150 additional calories, orange represents 151-200 additional calories, and red represents 201+ additional calories. It will be appreciated that these examples of color are intended to be illustrative rather than limiting, and that other combinations of color and/or brightness may be used in a comparable manner.

In an aspect of this embodiment, the wearable fitness device comprises a component to generate a dynamic lighting pulsation effect, wherein the brightness and pulse frequency decrease over time to visually represent the decay of the afterburn effect.

In an aspect of this embodiment, a component generates a post-workout metabolic report, such as a report to be made available in a companion application, and that displays the estimated afterburn duration (e.g., “2.3× metabolic rate for the next 18 hours”), a graphical representation of EPOC decay over time, and recommendations for optimizing afterburn in future workouts.

In an aspect of this embodiment, the wearable fitness device comprises means to permit a user to customize colors associated with estimated caloric afterburn ranges, and/or customize the estimated caloric afterburn ranges.

In an embodiment, a multi-surface display for a wearable fitness device comprises a wraparound, flexible, multi-surface display system configured to present distinct data sets across multiple display areas of the wearable band, with at least two configurable display surfaces and the option to incorporate additional display areas based on user preference or device configuration; a modular display structure that allows users to customize the number and arrangement of active display areas, ensuring adaptability for various training and biometric tracking needs. Each display surface is capable of presenting different performance metrics, including but not limited to heart rate, pace, cadence, speed, calorie expenditure, time in heart rate zones, post-workout metabolic activity (EPOC effects), exertion levels, recovery status, fatigue indicators, hydration status, respiration rate, stress levels, and other biometrically relevant data; a component to implement a user-configurable display setting that allows users to manually assign which metrics appear on each surface via a companion application, with the option for dynamic adjustments based on workout type, real-time performance, or historical trends; a wireless synchronization module that transmits the user's preferred display configuration to the wearable device via Bluetooth Low Energy (BLE) or an equivalent communication protocol; and a motion-sensitive orientation detection system that allows users to view different data points by naturally adjusting their wrist or arm position, without requiring manual screen navigation, button presses, or menu interactions.

In an embodiment, a method for user-configurable display customization in a wearable fitness device comprises providing a companion application that allows users to select, modify, and configure the arrangement of fitness data displayed on the wearable fitness device's multi-surface display; providing a component to implement a graphical user interface (GUI) presenting customizable display options, allowing users to assign specific data metrics to designated areas of the wearable fitness device; providing one or more predefined display configuration templates, wherein users can select from preset training modes (e.g., endurance running mode, HIIT mode, cycling mode) that auto-configure the display; and/or manually assign metrics to each display area to suit individual training preferences; and providing an automatic metric prioritization algorithm that suggests optimal metric placements based on historical user data and workout preferences, offering an AI-driven recommendation for personalized display settings.

In an embodiment, a smart magnetic clasp mechanism comprises a clasp-integrated power-on mechanism comprising multiple electrical contact points or alternative contact-based activation methods, including but not limited to magnetic, pressure-sensitive, touch-sensitive, or proximity-based mechanisms, ensuring power activation regardless of wrist or arm size; a size-adjustable clasp mechanism that maintains consistent metric visibility regardless of wrist circumference; and a low-energy wireless communication module, such as a Bluetooth module, that automatically establishes a connection with the user's fitness device when powered on. These embodiments may provide for power control and adaptive fit features, which may include, but are not necessarily limited to, automatic power on, automatic display compensation based on wrist or arm size, and automatic detection of band placement (e.g., automatic detection of whether the band is placed on the wrist or arm). These features may be integrated into other aspects of a wearable fitness device, for example by providing signals to indicate wrist circumference and/or band placement information.

In an embodiment, an adaptive display system that adjusts to wrist size comprises one or more components to perform sensor-based arm circumference detection and adaptive display by dynamically adjusting brightness, contrast, color output, or alternative visibility-enhancing mechanisms, and/or redistributing metric display positioning to maintain uniform visibility across different wrist and forearm sizes, wear positions, and ambient lighting conditions. The components further optimizes display orientation and clarity when the wearable fitness device is repositioned between wrist and arm placements. The components may also automatically adjusts light intensity and display clarity based on wrist/arm placement, ensuring uninterrupted metric readability and consistent user experience across different anatomical positions.

This embodiment of an adaptive display system may comprise a flexible lighting display strip that conforms to different wrist sizes while maintaining uniform light intensity; and one or more components to implement a software-based brightness balancing algorithm that prevents excessive brightness concentration on smaller wrists and ensures uniform illumination for all users.

In an embodiment, a method for providing a smart training benchmark and adaptive coaching system for performance optimization comprises implementing a historical performance analysis algorithm that continuously tracks and compares the user's current workout data to past sessions, dynamically adapts training benchmarks based on long-term progression trends, and generates real-time coaching prompts tailored to evolving fitness levels; providing a multi-modal AI coaching system that provides performance guidance through various feedback mechanisms. These may include, as non-limiting examples, Audible coaching cues (e.g., “Increase pace by 15 seconds per mile to match past performance”), haptic feedback through vibrations or rhythmic pulse patterns to indicate pacing adjustments, target heart rate zone adherence, or fatigue warnings; and visual coaching cues through dynamic color changes and pulsation effects on the wearable band, indicating whether the user is maintaining, exceeding, or falling behind their desired performance targets.

This embodiment may further comprise providing a customizable coaching mode that allows users to select preferred feedback styles (audio, haptic, visual, or a combination) based on training preferences and environmental factors (e.g., silent vibration feedback for race pacing, visual cues for treadmill training, or audio cues for outdoor running); and providing an AI-driven adaptive coaching engine that continuously refines feedback intensity, timing, and delivery based on user response patterns, ensuring optimal training support without excessive distractions.

It will be appreciated that aspects of these and other embodiments described herein may be combined in various ways, and that descriptions of an embodiment should not be construed in a way that limits that embodiment to one that only includes the specific features described.

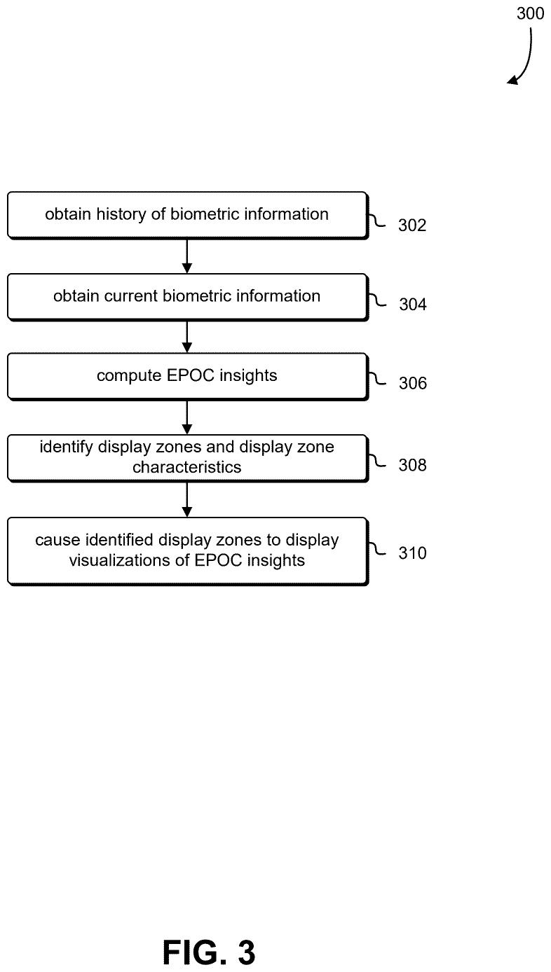

FIG. 3 illustrates an example 300 process of displaying EPOC information on a wearable fitness device, in accordance with an embodiment. Although FIG. 3 is depicted as a sequence of steps, embodiments may practice the disclosed method without necessarily adhering to the specific sequence that is depicted. For example, embodiment of the disclosed method may include steps or operations which are altered, reordered, or performed in parallel, except where logically required or explicitly indicated.

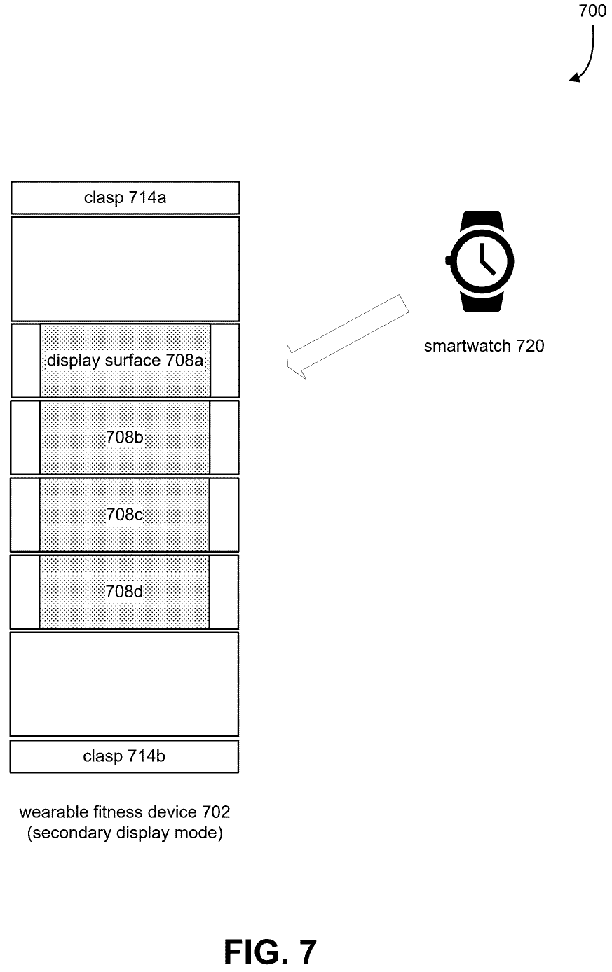

In some embodiments, the example process 300 of FIG. 3 is performed by a wearable fitness device, such as the wearable fitness device 102 depicted in FIG. 1, using at least one processor and memory comprising instructions that, when performed by the at least one processor, cause the device to perform the steps and operations of the depicted method. In other embodiments, aspects of the example process 300 are performed by another computing device, such as by the smartwatch 720 of FIG. 7, or by any other computing device or combination of computing devices. For example, in an embodiment, a smartwatch may be used to perform steps related to obtaining and processing exercise information, and the wearable fitness device may be used as a secondary display device that maximizes peripheral and/or off-axis perception of visual indicators.

At 302, the example process 300 obtains a history of biometric information for a user. In an embodiment, this comprises obtaining historical heart rate and exercise performance information.

At 304, the example process 300 obtains current biometric information for the user. In an embodiment, this comprises obtaining current (e.g., real-time during or after exercise) heart rate and exercise performance information.

At 306, the example process 300 computes EPOC insights. These may include insights into EPOC effects, such as insights into post-exercise afterburn effects, cardiac drift, or other insights, as described herein.

At 308, the example process 300 identifies display zones and display zone characteristics, as described herein, to determine where and how insights computed at step 306 should be displayed. This can comprise identifying display zones to maximize peripheral, off-axis, non-foveal perception by the user without altering the user's exercise performance.

At 310, the example process 300 causes these identified display zones to display visualizations of these EPOC insights.

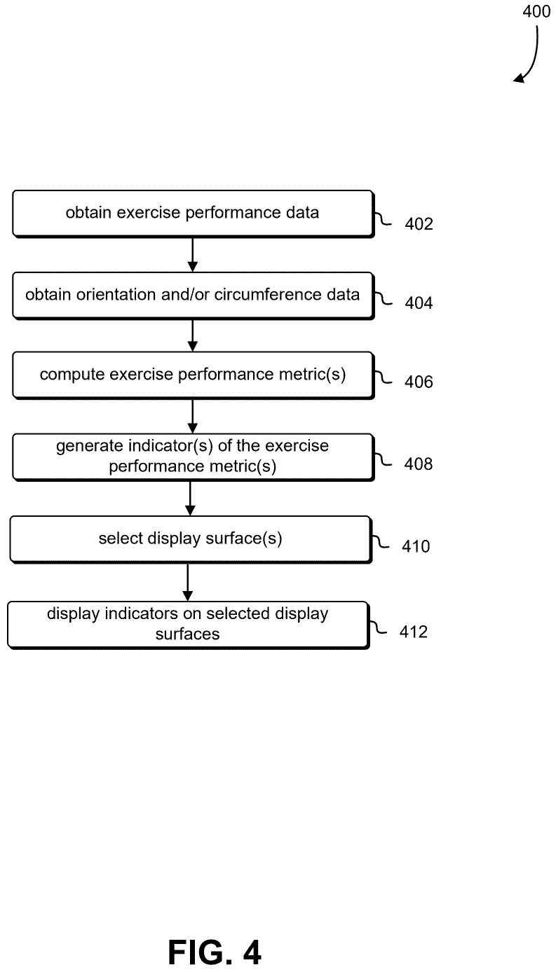

FIG. 4 illustrates an example process 400 of displaying exercise information on a flexible band, in accordance with an embodiment 400. Although FIG. 4 is depicted as a sequence of steps, embodiments may practice the disclosed method without necessarily adhering to the specific sequence that is depicted. For example, embodiment of the disclosed method may include steps or operations which are altered, reordered, omitted, or performed in parallel, except where logically required or explicitly indicated.

In some embodiments, the example process 400 of FIG. 4 is performed by a wearable fitness device, such as the wearable fitness device 102 depicted in FIG. 1, using at least one processor and memory comprising instructions that, when performed by the at least one processor, cause the device to perform the steps and operations of the depicted method. In other embodiments, aspects of the example process are performed by another computing device, such as by the smartwatch 720 of FIG. 7, or by any other computing device or combination of computing devices. For example, in an embodiment, a smartwatch or other computing device may be used to perform steps related to obtaining and processing exercise information, and the wearable fitness device may be used as a secondary display device that maximizes peripheral and/or off-axis perception of visual indicators.

At 402, the example process 400 obtains exercise performance data. This can comprise physiological or other information related to exercise performance. Examples of such data can include, but are not limited to data relating to heart rate, body temperature, electrocardiograms, heart rate variability, blood oxygen, body weight, other EPOC-related information, and so forth.

At 404, the example process 400 obtains orientation and/or circumference data. In an embodiment, this comprises obtaining information that indicates the circumference of the user's wrist or arm, or other data indicating the sizing and/or positioning of the wearable fitness device on the user's body. In some cases, the wearable fitness device determines positioning of the device relative to an average of the user's gaze during exercise.

At 406, the example process 400 computes exercise performance metrics. In an embodiment, these metrics include EPOC data, or other data related to the increased rate of oxygen intake following strenuous activity. This data may also be referred to as an afterburn or post-exercise effect data. Related metrics that may also be calculated can include effects on oxygen debt, metabolic rate, and caloric burn.

At 408, the example process 400 generates indicators of the exercise performance metrics. For example, the wearable fitness device may include software to generate visual indications that maximize user understanding of effects related to EPOC. The visualizations can include either relevant effects during exercise, and/or relevant effects after exercise. In embodiments, the visualizations are optimized for ambient and/or off-axis viewing. In some embodiments, this is achieved using circumference or orientation data, so that the generation of the visualization takes into account the position of the wearable fitness's device's relative to the user's average gaze during exercise.

At 410, the example process 400 selects one or more display surfaces on which to display the generated indicators. This may include selecting one or more display surfaces that maximize ambient and/or off-axis viewing, so that a visualization can be displayed on the device in a manner that can be perceived by the viewer using peripheral vision, or otherwise perceived without forcing the user to adjust the performance of an exercise. For example, by selecting a display surface that is positioned within the user's average gaze during jogging, a user can perceive displayed indications without altering their gate.

At 412, the example process 400 displays the indicators on the selected display surfaces. As described herein, the display surfaces and/or the visualizations themselves may be optimized so as to maximize ambient and/or off-axis viewing.

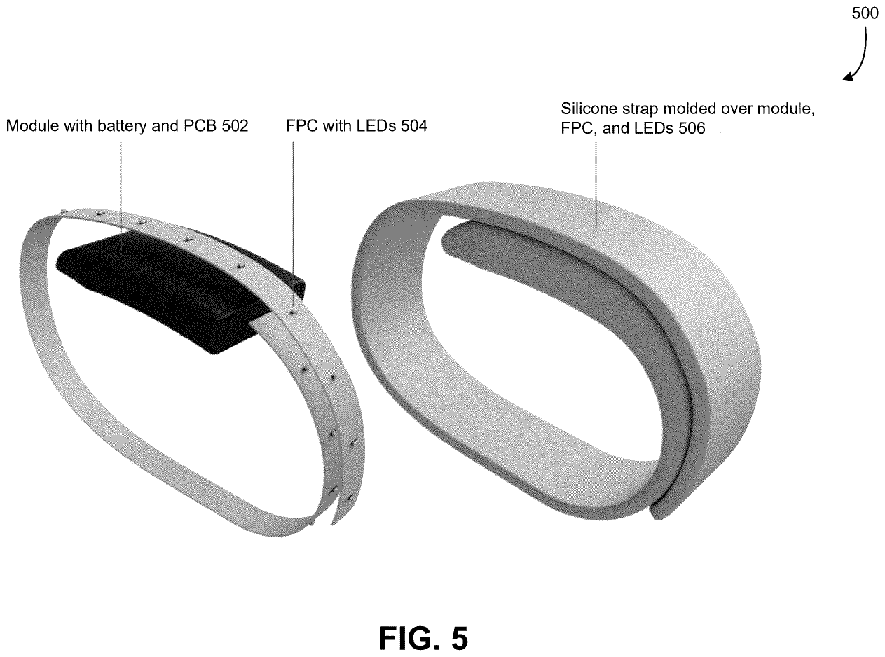

FIG. 5 illustrates an example embodiment of a wearable fitness device with a flexible band to display exercise information, in accordance with at least one embodiment. As depicted in the example 500, a wearable fitness device can comprise a module with battery and printed circuit board (PCB) 502 and a flexible printed circuit (FPC) with LEDs 504. The wearable fitness device may further comprise a silicone strap 506 that is molded over the module 502 and FPC with LEDs 504.

In an embodiment, the module with battery and PCB 502 comprises a processor, such as the processor 112 depicted in FIG. 1, that is printed or mounted on the PCB. The module 502 may further comprise non-transitory memory storing processor executable instructions that, when performed by the processor, cause the wearable fitness device to perform functionality described herein. The module 502 may further be coupled to the FPC with LEDs 504 to allow the processor to control the color and/or brightness of the LEDs.

The FPC with LEDs 504 may comprise a LEDs that correspond to a plurality of display surfaces. For example, each LED on the FPC with LEDs 504 may correspond to one of the display surfaces 104a-d, 106a-d, 108a-d, or physically proximate groups of the LEDs may each correspond to one of the display surfaces. In some embodiments, the number of display surfaces and the physical configuration of the display surfaces may be different than what is depicted in FIG. 1, and likewise the number and arrangement of the LEDs on the FPC may be different than what is depicted in FIG. 5.

The silicone strap 506 may be translucent, transparent, or semi-transparent at least in the locations of the LEDs, so illumination generated by the LEDs is visible on the user.

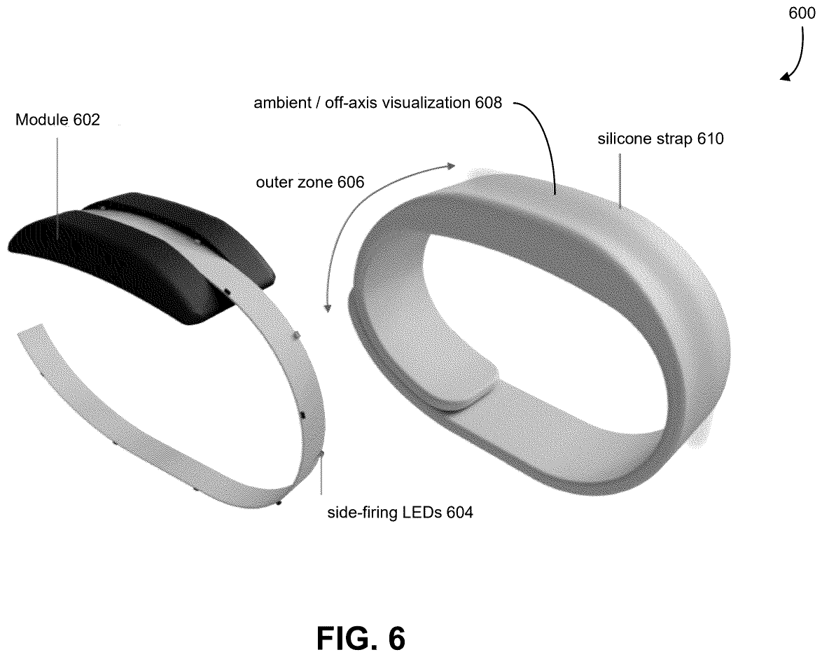

FIG. 6 illustrates an example embodiment of a wearable fitness device with a flexible band to display exercise information via an ambient or off-axis visualization, in accordance with at least one embodiment. In the example 600, a wearable fitness device comprises a module 602, side-firing LEDs 604, and silicone strap 610. These may correspond to the similarly named components 502, 504, 506 of FIG. 5.

In an embodiment of the example 600, the configuration of the LEDs 604 is such that it generates an ambient and/or off-axis visualization 608. The ambient and/or off-axis visualization 608 is a visualization generated by diffusing light to form a continuous visualization that is visible via peripheral vision (i.e., visible with off-axis viewing). This may remove the need for foveal focus (i.e., direct staring). Unlike standard displays on smartwatches, the ambient and/or off-axis visualization is optimized to offer maximum visibility of an exercise performance metric without requiring the user to break their ideal exercise form, such as their running form and/or gait.

In an embodiment, the wearable fitness device of example 600 includes an outer zone which may be free of LEDs. Alternatively, the outer zone 606 may include LEDs or other comparable displays that display additional or alternative visualizations.

FIG. 7 illustrates an example of a wearable fitness device to display exercise information as a secondary display, in accordance with an embodiment. In the example 700, a wearable fitness device 702 may comprise a plurality of display surfaces 708a-d. The display surfaces may be in any of various configurations, including but not limited to the configuration depicted in the wearable fitness device 102 of FIG. 1, or the simplified configuration depicted in FIG. 7. The wearable fitness device may also comprise a clasp mechanism 714a,b, similar to the clasp described in relation to FIG. 1.

The wearable fitness device 702 may operate as a secondary display to a smartwatch 720. A smartwatch may include, but is not limited to, smart watches by APPLE, GOOGLE, SAMSUNG, GARMIN, FITBIT, HUAWEI, and so forth. Alternatively and/or in addition, the wearable fitness device 720 may operate as a secondary display to other computing devices, including but not limited to smartphones, personal computers, cloud computing devices, and so forth. Although the example of FIG. 7 is explained using smartwatch 720, these other devices may be used in similar fashion.

In an embodiment, the wearable fitness device 720 includes one or more receivers to receive information from smartwatch 720, or other devices as noted above. In some embodiments, the received information may comprise low-bandwidth fitness data that is then processed by the wearable fitness device to generate a visual indication of exercise performance. In other embodiments, the wearable fitness device receives low-bandwidth indications of color and/or brightness to display in one or more selected display surfaces 708a-d. In still other embodiments, the wearable fitness device receives bitmaps, compressed video, or other higher-bandwidth image or video information from the smartwatch 720, where this information comprises a representation of a visual indication of exercise performance.

In some embodiments, the selection of display surfaces is determined by the smartwatch 720 and received by the wearable fitness device 702. In other embodiments, the wearable fitness device 702 selects which of the display surfaces to use. In some embodiments, selection of the display surface is made to maximize ambient and/or off-axis perception of the display by the user. For example, in at least one embodiment, the wearable fitness device uses circumference and/or positioning data to identify display surfaces that maximize ambient and/or off-axis perception of the display by the user. In some embodiments, the wearable fitness device 702 activates a display mode that maximizes ambient and/or off-axis perception of the display by the user. Examples of such display modes include use of side mounted display surfaces, the use of front-mounted display surfaces, or the activation and/or control of display surfaces likely to maximize ambient and/or off-axis perception during a particular kind of exercise or other activity. In at least one embodiment, the wearable fitness device 702 switches between maximizing ambient and/or off-axis perception during active exercise and minimizing intrusiveness of the display during a post-exercise period.

In at least one embodiment, a wearable fitness device comprises a band worn around a user's wrist or arm. The band may comprise one or more display surfaces configured to maximize perception by the wearer, particularly for off-axis viewing and scenarios where it is desirable to avoid the need for the user to rely on foveal vision to perceive a visual performance indicator. In at least one embodiment, the band is accompanied by a smartwatch that performs aspects of the processes described herein. In at least one embodiment, the band of the wearable fitness device is joined to or otherwise integrated with the smartwatch.

In at least one embodiment, the wearable fitness device provides maximized off-axis viewing of a runner's pace and heart rate.

In at least one embodiment, a user inputs desired splits into a software application, and the wearable fitness device generates visual indications (for example, by changing color and/or brightness) to indicate to the user whether they are on pace or not, according to the user-provided splits.

In at least one embodiment, heart rate and pace data is captured by a smartwatch that is communicatively coupled to the wearable fitness device. The wearable fitness device may be worn separately or be integrated with the smartwatch, for example as a band affixed to the smartwatch.

In at least one embodiment, a wearable fitness device comprises one or more of a microcontroller including a processor, a Bluetooth low-energy chip or other means of wireless communication with a smartwatch or other computing device, a battery, a charging port or charging interface, and one or more display surfaces. Embodiments of the one or more display surfaces are described herein.

In at least one embodiment, the wearable fitness device is configured to include diffusion material to cause light emitted from the display surfaces to be diffused. The diffusion material helps to diffuse the emitted light, so as to enable the emitted light to be more readily perceived off-axis, e.g. without the user needing to rely on foveal vision to perceive the corresponding visual indication.

In at least one embodiment, a smartwatch application runs in a background mode (e.g., leveraging an HKWorkoutSession and in a mode that enables receiving and sending Bluetooth and sensor information). The smartwatch application may further comprise a user interface to enable the user to enter exercise-related data, such as a target pace or split points.

In at least one embodiment, the smartwatch application determines applicable performance metrics and drives a display of the wearable fitness device. This can comprise causing the display surfaces of the wearable fitness device to display green when a metric is within a target range, red when the metric is above the target range, and blue when the metric is below the target range. It will be appreciated that other combinations of ranges and colors may be appropriate, and may be different for different types of exercise metrics.

In at least one embodiment, the smartwatch application may further include user interface elements to enable connection to the wearable fitness device. The application may also include user interface to set targets, such as target heart rate and pace. The application may also include means for detecting a current exercise state. In some cases this can be user interface elements that the user interacts with to indicate that they are exercising or have stopped exercising. Alternatively, smartwatch application can automatically detect this state using smartwatch features such as an accelerometer. The smartwatch application may also include user interface elements that can be interacted with to switch between different metrics to be indicated on the wearable fitness device. In some cases, different colors or groups of colors can be mapped to different exercise performance metrics. The user interface of the smartwatch application may include elements to display and or edit the association between exercise performance metrics and their corresponding colors, as well as various ranges associated with the different metrics and their visual indicators.

In at least one embodiment, the smartwatch application and/or wearable fitness device includes an audio mode, that can be used to provide context for visual signals without requiring glancing at the screen. For example, an audio alert may be generated when the colors displayed by the wearable fitness device change. For example, the audio alert might be a message such as “Pace too high. Slow down” to provide advise relevant to a displayed visual indication.

In at least one embodiment, a user can toggle between modes that reflect instant pace (e.g., within an exercise interval) and average pace (e.g., for marathon goal times).

In at least one embodiment, the wearable fitness device provides information related to how well a user adheres to a target goal. This may be referred to, in some embodiments, as a pulse score. For example, in one mode of operation, the wearable fitness device during exercise may indicate a green color when currently matching a target pace, and one or more other colors when outside the range. In another mode of operation, the wearable fitness device can display a simple score or color that corresponds to the percentage of time the user has spent in the “green” zone. This can assist the user in determining whether they conformed to the planned workout schedule.

In at least one embodiment, the wearable fitness device displays a smoothness metric. This can highlight volatility in metrics such as pace. In a smartwatch application, smoothness can be indicated in a visual display that includes a target band and highlights periods in which performance was inside, above, or below that band. On the wearable fitness device, a visual indicator can indicate green (for example) when metrics volatility is acceptably low.

In at least one embodiment, the smartwatch application can help visualize progression and trends in exercise performance. For example, the application may help visualize physiological progress by correlating Heart Rate cost against Pace output over time, and educate the user on why they are improving.

In at least one embodiment, post-run analysis and other information is provided using the smartwatch application and/or the wearable fitness device. The application, for example, can display a line graph overlaying pace and heart rate for a specific run. The application may also provide cardiac drift information, for example by highlights moments where pace remained steady but heart rate climbed, indicating fatigue or inefficiency. The application might also highlight periods where they stayed parallel, indicating aerobic efficiency.

In at least one embodiment, the application can also provide historical trend analysis. For example, the application can provide a chart showing average heart rate at an indicated pace, over selectable timeframes such as weeks or months. The application might also filter runs of similar intensity (e.g., “Tempo Runs”) to show the trend lines for each type of run.

In at least one embodiment, the application generates alerts or messages (e.g., a “milestone card”) when a user achieves a positive exercise trend. The milestone card can further include specific physiological explanations for the improvement, e.g. “Your heart is pumping more blood per beat,” “You're burning fuel more efficiently,” “Your consistent Zone 2 work has increased your Mitochondrial Density,” or “Oxygen is reaching your muscles faster. You have likely increased Capillary Density in your legs.” In at least one embodiment, these milestone cards are automatically sent or posted, e.g. to a social media account.

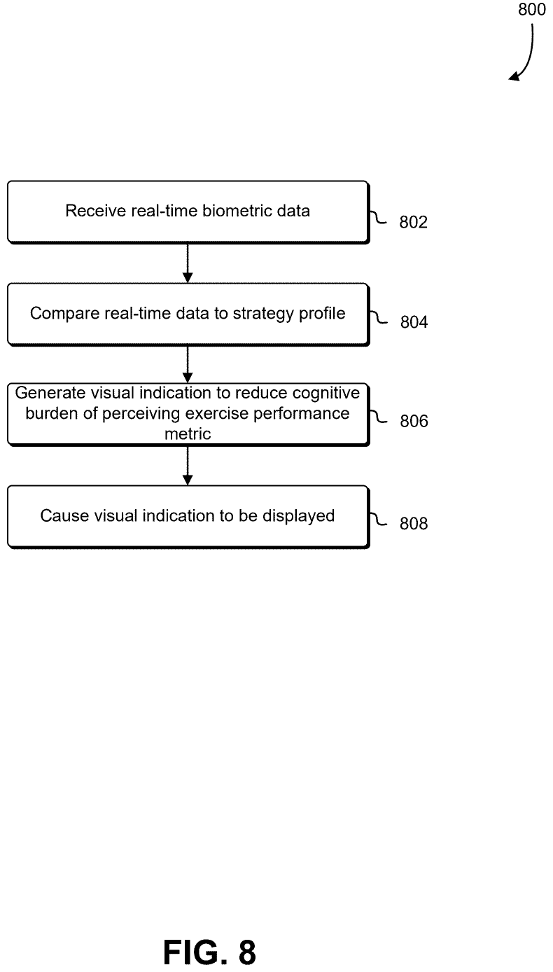

FIG. 8 illustrates an example process 800 of generating visual indications to reduce the cognitive burden of perceiving an exercise performance metric. Although FIG. 8 is depicted as a sequence of steps, embodiments may practice the disclosed method without necessarily adhering to the specific sequence that is depicted. For example, embodiment of the disclosed method may include steps or operations which are altered, reordered, omitted, or performed in parallel, except where logically required or explicitly indicated.

In some embodiments, the example process 800 of FIG. 8 is performed by a wearable fitness device, such as the wearable fitness device 102 depicted in FIG. 1, using at least one processor and memory comprising instructions that, when performed by the at least one processor, cause the device to perform the steps and operations of the depicted method. In other embodiments, aspects of the example process 800 are performed by another computing device, such as by the smartwatch 720 of FIG. 7, or by any other computing device or combination of computing devices. For example, in an embodiment, a smartwatch may be used to perform steps related to obtaining and processing exercise information, and the wearable fitness device may be used as a secondary display device that maximizes peripheral and/or off-axis perception of visual indicators.

At 802, the example process 800 receives real-time biometric data. This can include, for example, heart rate, body temperature, electrocardiogram data, and other such information. The information may also include exercise metrics such as pace or other kinetic information. In some embodiments, biometric data and/or exercise metrics are obtained from biometric sensors included in the wearable fitness device. In other embodiments, the biometric data and/or exercise metrics are obtained from another computing device, such as the smartwatch 720 depicted in FIG. 7.

At 804, the example process 800 compares real-time data to a strategy profile. In at least one embodiment, a strategy profile comprises information detailing a strategy for maximizing exercise performance benefits. The strategy profile may be generated, in some cases, on a computing device such as a smartphone, smartwatch, personal computer, web service, and so forth. In some cases, the strategy profile is then downloaded to the wearable fitness device.

In some embodiments, the strategy profile indicates ranges of performance metrics that are indicated as ideal for a type of exercise or exercise goal.

At 806, the example process 800 generates a visual indication that reduces the cognitive burden of perceiving an exercise performance metric. This may be accomplished using the range information indicated in the strategy profile. For example, in at least one embodiment, a simplified state signal may be generated to represent the current value of an exercise performance metric. A blue color indication may be generated to indicate the metric being below target, a green color indication may be generated to indicate the metric being on target, and a red color indication may be generated to indicate the metric being above target.

At 808, the example process 800 causes the visual indication to be displayed. In some embodiments, the visual indication is displayed on display surfaces configured and/or selected to maximize ambient and/or off-axis perception of the visual indication by the user wearing the fitness device.

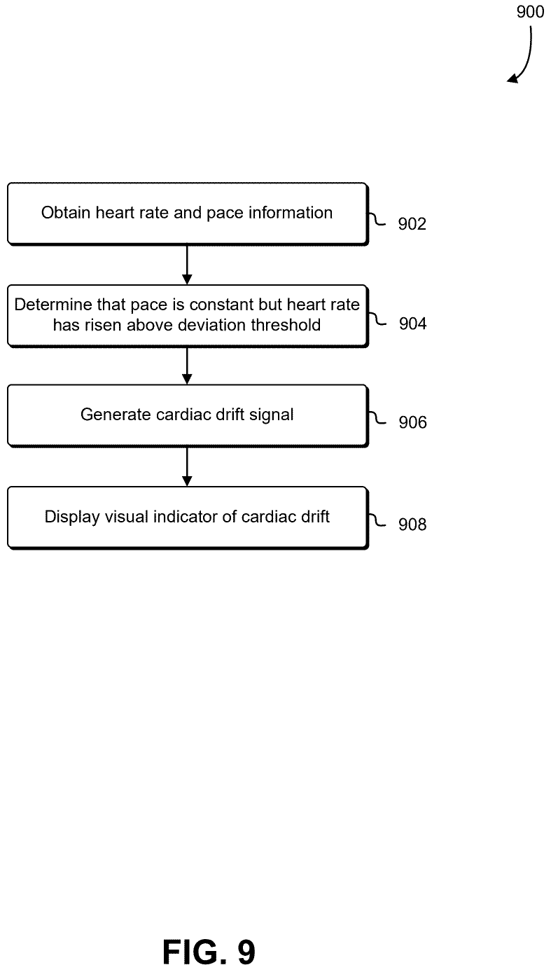

FIG. 9 illustrates an example process 900 of generating a visual indication of an exercise performance metric, in accordance with an embodiment. Although FIG. 9 is depicted as a sequence of steps, embodiments may practice the disclosed method without necessarily adhering to the specific sequence that is depicted. For example, embodiment of the disclosed method may include steps or operations which are altered, reordered, omitted, or performed in parallel, except where logically required or explicitly indicated.

In some embodiments, the example process 900 of FIG. 9 is performed by a wearable fitness device, such as the wearable fitness device 102 depicted in FIG. 1, using at least one processor and memory comprising instructions that, when performed by the at least one processor, cause the device to perform the steps and operations of the depicted method. In other embodiments, aspects of the example process 900 are performed by another computing device, such as by the smartwatch 720 of FIG. 7, or by any other computing device or combination of computing devices. For example, in an embodiment, a smartwatch may be used to perform steps related to obtaining and processing exercise information, and the wearable fitness device may be used as a secondary display device that maximizes peripheral and/or off-axis perception of visual indicators.

At 902, the example process 900 obtains heart rate and pace information. As described herein, this information may be obtained using sensors on the wearable fitness device, or from a computing device such as the smartwatch 720 depicted in FIG. 7. In embodiments, heart rate refers to the speed at which the user's heart is beating during performance of an exercise, and pace refers to a metric such as distance over time, steps over time, and so forth that are relevant to the user's exercise.

At 904, the example process 900 determines that pace is constant but heart rate has risen above a deviation threshold. In some embodiments, a constant pace is determined when a metric indicative of pace remains within a pace deviation threshold over a given period of time. Similarly, heart rate may be determined to be constant when within a heart rate deviation threshold over a given period of time, or not constant when outside that threshold.

At 906, the example process 900 generates a cardiac drift signal. In embodiments, the heart rate deviation being above a heart rate deviation threshold is used to determine that the user has entered a cardiac drift state. Cardiac drift can refer to a state in which heart rate rises during a prolonged period of exercise, and can be caused, for example, by dehydration and increased core body temperature. The process 906 may therefore respond to this detection by generating a cardiac drift signal.

At 908, the example process 900 displays a visual indicator of cardiac drift. The visual indicator can be generated, as described herein, in a manner that optimizes for peripheral and/or off-axis viewing, so that the user of the wearable fitness device can perceive the indicator without altering their gate or other aspects of performing the exercise.

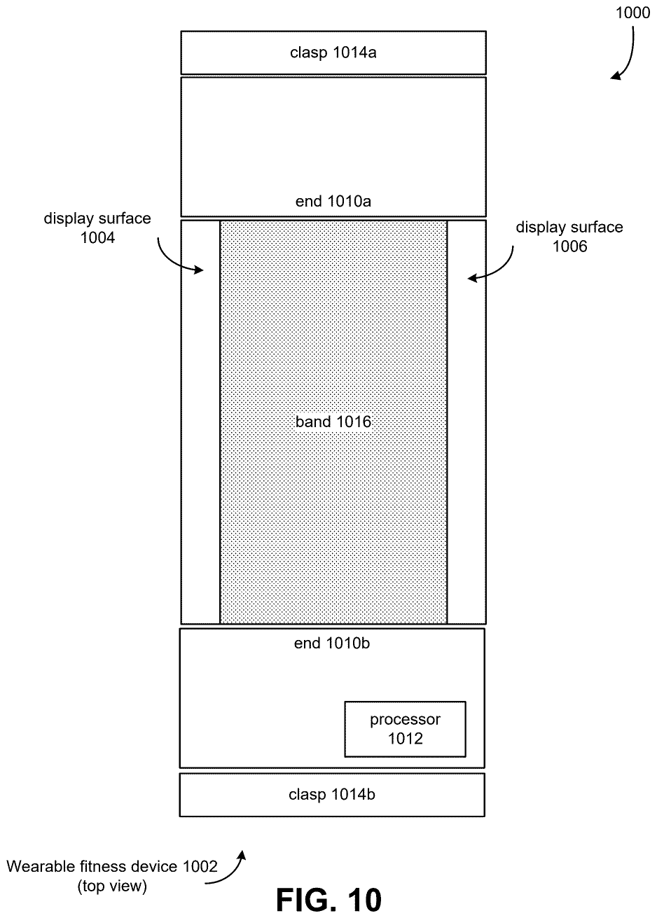

FIG. 10 illustrates aspects of a wearable fitness device to display exercise information using side-oriented display surfaces, in accordance with an embodiment. In the example 1000, the wearable fitness device 1002 may comprise clasps 1014a,b and ends 1010a,b corresponding to the clasps 114a,b and ends 110a,b of FIG. 1. The device may also comprise one or more display surfaces 1004, 1006 that are continuous along each edge of a band 1016. In some embodiments, the central portion of the band 1016 has no display surface, although other embodiments may have one or more display surfaces in this location. The wearable fitness device 1002 may also comprise a processor 1012, which can in some embodiments correspond to the processor 112 depicted in FIG. 1. In at least one embodiment, wearable fitness device 102 is a watch band that is affixed to a smartwatch, such as the smartwatch 720 depicted in FIG. 7. In other embodiments, the wearable fitness device 1002 is a separate device that works in cooperation with or independently of a smartwatch, as described herein with respect to various embodiments.

In an embodiment, the display surfaces function as a peripheral optical band or ambient optical interface, meaning that they are configured to diffuse light into a continuous visualization that is easily visible via peripheral, off-axis, or non-foveal vision. The diffusion may be accomplished by using one or more techniques such as embedded scattering particles, volume diffusion, surface texturing, surface-relief, and transmission through translucent materials. For example, in an embodiment, the display surfaces 1004, 1006 comprise a light source, such as an LED, that is transmitted through a material to generate a diffuse lighting effect that is prominently visible to the user. In an embodiment, the material is a transparent, semi-transparent, or translucent silicone band. In other embodiments, a glass or acrylic material is used. In other embodiments, surface texturing on the band 1016 causes light diffusion to emphasize a visual indication on a portion of the band that is perceptible via peripheral, off-axis, or non-foveal vision.

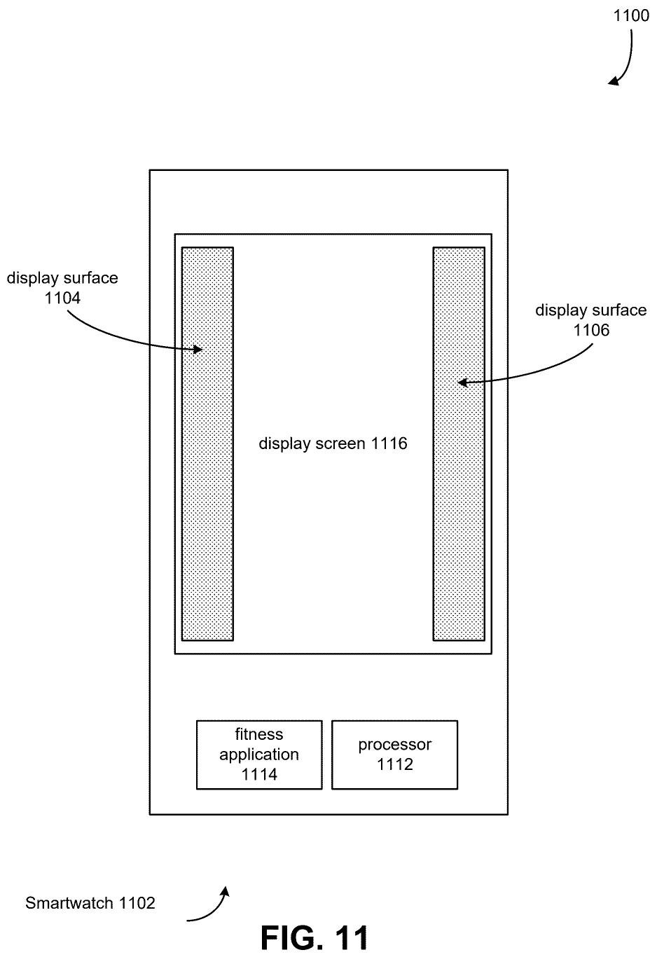

FIG. 11 illustrates a software application to generate visual signals for non-foveal perception on a smartwatch. In this embodiment 1100, a smartwatch 1102 comprises a processor 1112 that performs processor executable instructions corresponding to a fitness application 1114. The fitness application may assign portions of a display screen 1116 to serve as display surfaces 1106. These may be positioned on the smartwatch's display screen 116 so as to maximize non-foveal (i.e., peripheral or off-axis) perception of visual indicators. Techniques described herein, for example the techniques described in relation to any of FIGS. 1-10, may be performed using these software-driven display surfaces, realized on display screen 1116, instead of display surfaces such as independent LEDs.

In some embodiments, non-foveal perception is maximized by periodic increase or decrease of a visual indications brightness. This may be further enhanced by timing the pulses in a manner that aligns with the user's gait, or that is positioned so maximize perception. For example, the fitness application might select only one of two of the display surfaces 1104, 1106 to activate based on whether the smartwatch is being worn on the left or right hand, so that the indication appears on the side most likely to be in the user's peripheral vision during a normal running gait. Note that this technique for selecting a display surface may also be applied to other embodiments, e.g. those described in relation to any of FIGS. 1-10. One benefit of this technique may be that it preserves battery life by limiting energy expended to create a bright display to only those areas that are most likely to be perceived by the user in their normal conduct of exercise.

Note that, in the context of describing disclosed embodiments, unless otherwise specified, use of expressions regarding executable instructions (also referred to as code, applications, agents, etc.) performing operations that “instructions” do not ordinarily perform unaided (e.g., transmission of data, calculations, etc.) denotes that the instructions are being executed by a machine, thereby causing the machine to perform the specified operations.

One or more computing devices may be used to practice embodiments of the present disclosure. In various embodiments, a computing device includes any appropriate device operable to send and/or receive requests, messages, or information over an appropriate network and convey information back to a user of the device. The computing device may be used to implement any of the systems illustrated and described above. For example, the computing device may be configured for use as a data server, a web server, a portable computing device, a personal computer, a cellular or other mobile phone, a handheld messaging device, a laptop computer, a tablet computer, a set-top box, a personal data assistant, an embedded computer system, an electronic book reader, or any electronic computing device. The computing device may be implemented as a hardware device, a virtual computer system, or one or more programming modules executed on a computer system, and/or as another device configured with hardware and/or software to receive and respond to communications (e.g., web service application programming interface (API) requests) over a network. Aspects of the computing device described herein may also be incorporated, where appropriate, into embodiments of a wearable fitness device as described herein.

The computing device may include one or more processors that, in embodiments, communicate with and are operatively coupled to a number of peripheral subsystems via a bus subsystem. In some embodiments, these peripheral subsystems include a storage subsystem comprising a memory subsystem and a file/disk storage subsystem, one or more user interface input devices, one or more user interface output devices, and a network interface subsystem. Such storage subsystem may be used for temporary or long-term storage of information.

In some embodiments, the bus subsystem may provide a mechanism for enabling the various components and subsystems of computing device to communicate with each other as intended. Although the bus subsystem is shown schematically as a single bus, alternative embodiments of the bus subsystem utilize multiple buses. The network interface subsystem may provide an interface to other computing devices and networks. The network interface subsystem may serve as an interface for receiving data from and transmitting data to other systems from the computing device. In some embodiments, the bus subsystem is utilized for communicating data such as details, search terms, and so on. In an embodiment, the network interface subsystem may communicate via any appropriate network that would be familiar to those skilled in the art for supporting communications using any of a variety of commercially available protocols, such as Transmission Control Protocol/Internet Protocol (TCP/IP), User Datagram Protocol (UDP), protocols operating in various layers of the Open System Interconnection (OSI) model, File Transfer Protocol (FTP), Universal Plug and Play (UpnP), Network File System (NFS), Common Internet File System (CIFS), and other protocols.

The network, in an embodiment, is a local area network, a wide-area network, a virtual private network, the Internet, an intranet, an extranet, a public switched telephone network, a cellular network, an infrared network, a wireless network, a satellite network, or any other such network and/or combination thereof, and components used for such a system may depend at least in part upon the type of network and/or system selected. In an embodiment, a connection-oriented protocol is used to communicate between network endpoints such that the connection-oriented protocol (sometimes called a connection-based protocol) is capable of transmitting data in an ordered stream. In an embodiment, a connection-oriented protocol can be reliable or unreliable. For example, the TCP protocol is a reliable connection-oriented protocol. Asynchronous Transfer Mode (ATM) and Frame Relay are unreliable connection-oriented protocols. Connection-oriented protocols are in contrast to packet-oriented protocols such as UDP that transmit packets without a guaranteed ordering. Many protocols and components for communicating via such a network are well known and will not be discussed in detail. In an embodiment, communication via the network interface subsystem is enabled by wired and/or wireless connections and combinations thereof.

In some embodiments, the user interface input devices includes one or more user input devices such as a keyboard; pointing devices such as an integrated mouse, trackball, touchpad, or graphics tablet; a scanner; a barcode scanner; a touch screen incorporated into the display; audio input devices such as voice recognition systems, microphones; and other types of input devices. In general, use of the term “input device” is intended to include all possible types of devices and mechanisms for inputting information to the computing device. In some embodiments, the one or more user interface output devices include a display subsystem, a printer, or non-visual displays such as audio output devices, etc. In some embodiments, the display subsystem includes a cathode ray tube (CRT), a flat-panel device such as a liquid crystal display (LCD), light emitting diode (LED) display, or a projection or other display device. In general, use of the term “output device” is intended to include all possible types of devices and mechanisms for outputting information from the computing device. The one or more user interface output devices can be used, for example, to present user interfaces to facilitate user interaction with applications performing processes described and variations therein, when such interaction may be appropriate.

In some embodiments, the storage subsystem provides a computer-readable storage medium for storing the basic programming and data constructs that provide the functionality of at least one embodiment of the present disclosure. The applications (programs, code modules, instructions), when executed by one or more processors in some embodiments, provide the functionality of one or more embodiments of the present disclosure and, in embodiments, are stored in the storage subsystem. These application modules or instructions can be executed by the one or more processors. In various embodiments, the storage subsystem additionally provides a repository for storing data used in accordance with the present disclosure. In some embodiments, the storage subsystem comprises a memory subsystem and a file/disk storage subsystem.

In embodiments, the memory subsystem includes a number of memories, such as a main random-access memory (RAM) for storage of instructions and data during program execution and/or a read only memory (ROM), in which fixed instructions can be stored. In some embodiments, the file/disk storage subsystem provides a non-transitory persistent (non-volatile) storage for program and data files and can include a hard disk drive, a floppy disk drive along with associated removable media, a Compact Disk Read Only Memory (CD-ROM) drive, an optical drive, removable media cartridges, or other like storage media.

The computing device could be of any of a variety of types, including a portable computer device, tablet computer, a workstation, or any other device described below. Additionally, the computing device can include another device that, in some embodiments, can be connected to the computing device through one or more ports (e.g., USB, a headphone jack, Lightning connector, etc.). Due to the ever-changing nature of computers and networks, this description of the computing device is intended only as a specific example for purposes of illustrating embodiments of such a device.

The specification and drawings are, accordingly, to be regarded in an illustrative rather than a restrictive sense. However, it will be evident that various modifications and changes may be made thereunto without departing from the scope of the invention as set forth in the claims. Likewise, other variations are within the scope of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed but, on the contrary, the intention is to cover all modifications, alternative constructions and equivalents falling within the scope of the invention, as defined in the appended claims.

In some embodiments, data may be stored in a data store (not depicted). In some examples, a “data store” refers to any device or combination of devices capable of storing, accessing, and retrieving data, which may include any combination and number of data servers, databases, data storage devices, and data storage media, in any standard, distributed, virtual, or clustered system. A data store, in an embodiment, communicates with block-level and/or object level interfaces. The computing device may include any appropriate hardware, software, and firmware for integrating with a data store as needed to execute aspects of one or more applications for the computing device to handle some or all of the data access and business logic for the one or more applications. The data store, in an embodiment, includes several separate data tables, databases, data documents, dynamic data storage schemes, and/or other data storage mechanisms and media for storing data relating to a particular aspect of the present disclosure. In an embodiment, the computing device includes a variety of data stores and other memory and storage media as discussed above. These can reside in a variety of locations, such as on a storage medium local to (and/or resident in) one or more of the computers or remote from any or all of the computers across a network. In an embodiment, the information resides in a storage-area network (SAN) familiar to those skilled in the art, and, similarly, any necessary files for performing the functions attributed to the computers, servers or other network devices are stored locally and/or remotely, as appropriate.

In an embodiment, the computing device may provide access to content including, but not limited to, text, graphics, audio, video, and/or other content that is provided to a user in the form of HyperText Markup Language (HTML), Extensible Markup Language (XML), JavaScript, Cascading Style Sheets (CSS), JavaScript Object Notation (JSON), and/or another appropriate language. The computing device may provide the content in one or more forms including, but not limited to, forms that are perceptible to the user audibly, visually, and/or through other senses. The handling of requests and responses, as well as the delivery of content, in an embodiment, is handled by the computing device using PHP: Hypertext Preprocessor (PHP), Python, Ruby, Perl, Java, HTML, XML, JSON, and/or another appropriate language in this example. In an embodiment, operations described as being performed by a single device are performed collectively by multiple devices that form a distributed and/or virtual system.

In an embodiment, the computing device typically will include an operating system that provides executable program instructions for the general administration and operation of the computing device and includes a computer-readable storage medium (e.g., a hard disk, random access memory (RAM), read only memory (ROM), etc.) storing instructions that if executed (e.g., as a result of being executed) by a processor of the computing device cause or otherwise allow the computing device to perform its intended functions (e.g., the functions are performed as a result of one or more processors of the computing device executing instructions stored on a computer-readable storage medium).

In an embodiment, the computing device operates as a web server that runs one or more of a variety of server or mid-tier applications, including Hypertext Transfer Protocol (HTTP) servers, FTP servers, Common Gateway Interface (CGI) servers, data servers, Java servers, Apache servers, and business application servers. In an embodiment, computing device is also capable of executing programs or scripts in response to requests from user devices, such as by executing one or more web applications that are implemented as one or more scripts or programs written in any programming language, such as Java®, C, C #or C++, or any scripting language, such as Ruby, PHP, Perl, Python, or TCL, as well as combinations thereof. In an embodiment, the computing device is capable of storing, retrieving, and accessing structured or unstructured data. In an embodiment, computing device additionally or alternatively implements a database, such as one of those commercially available from Oracle®, Microsoft®, Sybase®, and IBM® as well as open-source servers such as MySQL, Postgres, SQLite, MongoDB. In an embodiment, the database includes table-based servers, document-based servers, unstructured servers, relational servers, non-relational servers, or combinations of these and/or other database servers.

The use of the terms “a” and “an” and “the” and similar referents in the context of describing the disclosed embodiments (especially in the context of the following claims) is to be construed to cover both the singular and the plural, unless otherwise indicated or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to, or joined together, even if there is something intervening. Recitation of ranges of values in the present disclosure are merely intended to serve as a shorthand method of referring individually to each separate value falling within the range unless otherwise indicated and each separate value is incorporated into the specification as if it were individually recited. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal. The use of the phrase “based on,” unless otherwise explicitly stated or clear from context, means “based at least in part on” and is not limited to “based solely on.”

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., could be either A or B or C, or any nonempty subset of the set of A and B and C. For instance, in the illustrative example of a set having three members, the conjunctive phrases “at least one of A, B, and C” and “at least one of A, B, and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

Operations of processes described can be performed in any suitable order unless otherwise indicated or otherwise clearly contradicted by context. Processes described (or variations and/or combinations thereof) can be performed under the control of one or more computer systems configured with executable instructions and can be implemented as code (e.g., executable instructions, one or more computer programs or one or more applications) executing collectively on one or more processors, by hardware or combinations thereof. In some embodiments, the code can be stored on a computer-readable storage medium, for example, in the form of a computer program comprising a plurality of instructions executable by one or more processors. In some embodiments, the computer-readable storage medium is non-transitory.

The use of any and all examples, or exemplary language (e.g., “such as”) provided, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described, including the best mode known to the inventors for carrying out the invention. Variations of those embodiments will become apparent to those of ordinary skill in the art upon reading the foregoing description. The inventors expect skilled artisans to employ such variations as appropriate, and the inventors intend for embodiments of the present disclosure to be practiced otherwise than as specifically described. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated or otherwise clearly contradicted by context.

All references, including publications, patent applications, and patents, cited are hereby incorporated by reference to the same extent as if each reference were individually and specifically indicated to be incorporated by reference and were set forth in its entirety.

What is claimed is:

1. A device, comprising:
a flexible band conforming to size and dimensions of at least one of a wrist or arm, wherein the flexible band comprises at least one display surface spanning a proportion of the flexible band;
one or more processors to:
obtain exercise performance data corresponding to a user of the device;
compute an exercise performance metric based, at least in part, on the exercise performance data; and
cause the at least one display surface to display one or more indicators of the exercise performance metric, wherein the indicator comprises at least one of a color and/or brightness corresponding to a range of the exercise performance metric.

2. The device of claim 1, wherein the exercise performance metric corresponds to at least one of heart rate, pace, cadence, post-exercise oxygen consumption, or cardiac drift.

3. The device of claim 1, wherein the one or more indicators comprises a first color and/or brightness when caloric afterburn falls within a first range and wherein the one or more indicators comprise a second color and/or brightness when caloric afterburn falls within a second range different than the first range.

4. The device of claim 1, wherein the one or more processors are to:
compute excess post-exercise oxygen consumption (EPOC); and
cause the at least one display surface to display the one or more indicators based, at least in part, on the computed EPOC.

5. The device of claim 1, wherein the one or more processors are further to:
determine a physical orientation of the device; and
select the at least one display surface based, at least in part, on the physical orientation.

6. The device of claim 1, wherein the one or more processors are further to cause the color and/or brightness of the one or more indicators to change over time to indicate a change to the exercise performance metric.

7. The device of claim 1, wherein the one or more processors are to generate one or more signals to indicate one or more instructions to the user to alter performance of an exercise.

8. The device of claim 1, wherein light emitted by the at least one display surface is diffused to maximize non-foveal perception.

9. The device of claim 1, wherein the one or more processors are to:
determine that a user's exercise pace is within a first deviation threshold and that the user's heart rate is above a second deviation threshold; and
in response to the determination, display the one or more indicators to indicate cardiac drift.

10. The device of claim 1, wherein the one or more processors are to:
determine that the exercise performance metric is one of within, above, or below a target range; and
cause the one or more indicators to include a color indicator corresponding the determination.

11. The device of claim 1, wherein the flexible band comprises a plurality of display surfaces including the at least one display surface, and wherein each display surface is configured to display a different at least one indicator.

12. A wearable fitness device, comprising:
a plurality of display surfaces;
one or more processors to:
obtain information indicative of a selection of one or more of the plurality of display surfaces to activate, wherein the selection is based, at least in part, on maximization of non-foveal perception of the display by a user wearing the wearable fitness device;
obtain information indicative of an exercise performance metric of the user; and
cause a visual indication corresponding to the information indicative of the exercise to be displayed on the selected ones of the plurality of display surfaces.

13. The wearable fitness device of claim 12, wherein the information indicative of the exercise performance metric is obtained from another computing device.

14. The wearable fitness device of claim 12, wherein the one or more processors compute the selection of the one or more of the plurality of display surfaces.

15. The wearable fitness device of claim 12, further comprising a clasp mechanism to generate one or more indications used by the one or more processors to select the one or more of the plurality of display surfaces.

16. The wearable fitness device of claim 12, wherein the plurality of display surfaces generate light that is diffused to maximize non-foveal perception.

17. The wearable fitness device of claim 12, wherein the selection of the one or more of the plurality of display surfaces is based, at least in part, on at least one of a detected circumference or position of the wearable fitness device as worn by the user.

18. The wearable fitness device of claim 12, wherein the visual indication comprises a first color and/or brightness when caloric afterburn falls within a first range and wherein the visual indication comprise a second color and/or brightness when caloric afterburn falls within a second range different than the first range.

19. The wearable fitness device of claim 12, wherein the visual indication reduces metric data received from a computing device to maximize non-foveal perception of an exercise performance characteristic corresponding to the metric data.

20. A method implemented by one or more processors, comprising:
obtaining exercise performance data corresponding to a user of a wearable fitness device;
computing an exercise performance metric based, at least in part, on the exercise performance data; and
causing at least one display surface to display one or more indicators of the exercise performance metric, wherein the one or more indicators comprises at least one of a color and/or brightness corresponding to a range of the exercise performance metric, and wherein the one or more indicators are perceptible using non-foveal vision based, at least in part, on one or more characteristics of the at least one display surface.

21. The method of claim 20, wherein the one or more characteristics of the at least one display surface comprises light transmission through a diffusive medium.

22. The method of claim 20, wherein the one or more characteristics of the at least one display surface comprises a location on the wearable fitness device that is visible by a wearer of the wearable fitness device during exercise using non-foveal vision.

23. The method of claim 20, further comprising selecting the at least one display surface, from a plurality of display surfaces, by determining that the selected at least one display surface will maximize non-foveal perception of the one or more indicators.

24. The method of claim 20, wherein the one or more indicators comprise a first color and/or brightness when caloric afterburn falls within a first range and wherein the one or more indicators comprise a second color and/or brightness when caloric afterburn falls within a second range different than the first range.

25. The method of claim 20, further comprising:
computing excess post-exercise oxygen consumption (EPOC) based, at least in part, on the exercise performance data; and
causing the at least one display surface to display the one or more indicators based, at least in part, on the computed EPOC.

26. The method of claim 20, further comprising:
determining that a user's exercise pace is within a first deviation threshold and that the user's heart rate is above a second deviation threshold; and
generating, in response to the determining, the one or more indicators to include an indicator of cardiac drift.
